(19) United States
(12) Patent Application Publication
(10) Pub. No.: US 2026/0041651 A1
(43) Pub. Date: Feb. 12, 2026

(54) BUPROPION AND DEXTROMETHORPHAN FOR REDUCTION OF SUICIDE RISK IN DEPRESSION PATIENTS

(71) Applicant: ANTECIP BIOVENTURES II LLC, New York, NY (US)

(72) Inventor: Herriot Tabuteau, New York, NY (US)

(21) Appl. No.: 19/278,703

(22) Filed: Jul. 23, 2025

Related U.S. Application Data
(63) Continuation of application No. 18/397,220, filed on Dec. 27, 2023, now Pat. No. 12,390,428.
Application No. 18/397,220 is a continuation of application No. 18/323,714, filed on May 25, 2023, now Pat. No. 11,896,563.
Application No. 18/323,714 is a continuation of application No. PCT/US2021/061492, filed on Dec. 1, 2021.
(60) Provisional application No. 63/120,160, filed on Dec. 1, 2020.
(60) Provisional application No. 63/120,672, filed on Dec. 2, 2020.
(60) Provisional application No. 63/122,902, filed on Dec. 8, 2020.

(51) Int. Cl. A61K 31/137; A61K 31/485; A61P 25/24

(52) U.S. Cl. CPC A61K 31/137; A61K 31/485; A61P 25/24

(57) ABSTRACT

This disclosure relates to a method of treating depression and/or reducing risk of suicide, comprising administering a combination of about 90 mg to about 120 mg of bupropion hydrochloride, or a molar equivalent amount of another form of bupropion, and about 40 mg to about 50 mg of dextromethorphan hydrobromide, or a molar equivalent amount of another form of dextromethorphan. The combination may be administered twice a day to a human being suffering from major depressive disorder and having a score of 3 or greater on the Suicidality Item of the Montgomery-Åsberg Depression Rating Scale (MADRS-SI).

### CROSS-REFERENCE TO RELATED APPLICATIONS

This application is a continuation of U.S. patent application Ser. No. 18/397,220, filed Dec. 27, 2023; which is a continuation of U.S. patent application Ser. No. 18/323,714, filed May 25, 2023, now U.S. Pat. No. 11,896,563; which is a continuation of International Pat. App. No. PCT/US2021/061492, filed Dec. 1, 2021; which claims the benefit of U.S. Provisional App. Nos. 63/120,160, filed Dec. 1, 2020, 63/120,672, filed Dec. 2, 2020, and 63/122,902, filed Dec. 8, 2020; all of which are incorporated by reference herein in their entireties.

### SUMMARY

This disclosure relates to a method of treating depression and/or reducing risk of suicide, comprising administering combination of about 90 mg to about 120 mg of bupropion hydrochloride, or a molar equivalent amount of another form of bupropion, and about 40 mg to about 50 mg of dextromethorphan hydrobromide, or a molar equivalent amount of another form of dextromethorphan. The combination may be administered twice a day to a human being suffering from major depressive disorder and having a score of 3 or greater on the Suicidality Item of the Montgomery-Åsberg Depression Rating Scale (MADRS-SI).

### DETAILED DESCRIPTION

The combination of dextromethorphan and bupropion may be used to treat depression, such as major depressive disorder and/or reduce the risk of suicide. Patients being treated by this combination may suffer from depression and may have a score of 2 or greater, or 3 or greater on the Suicidality Item of the Montgomery-Åsberg Depression Rating Scale (MADRS-SI).

Dextromethorphan is rapidly metabolized in the human liver. This rapid hepatic metabolism may limit systemic drug exposure in individuals who are extensive metabolizers. Human beings can be: 1) extensive metabolizers of dextromethorphan—those who rapidly metabolize dextromethorphan; 2) poor metabolizers of dextromethorphan-those who only poorly metabolize dextromethorphan; or 3) intermediate metabolizers of dextromethorphan-those whose metabolism of dextromethorphan is somewhere between that of an extensive metabolizer and a poor metabolizer. Extensive metabolizers can also be ultra-rapid metabolizers. Non-poor metabolizers of dextromethorphan include extensive metabolizers of dextromethorphan and intermediate metabolizers of dextromethorphan. Extensive metabolizers of dextromethorphan are a significant portion of the human population. Dextromethorphan can, for example, be metabolized to dextrorphan.

When given the same oral dose of dextromethorphan, plasma levels of dextromethorphan are significantly higher in poor metabolizers or intermediate metabolizers as compared to extensive metabolizers of dextromethorphan. The low plasma concentrations of dextromethorphan can limit its clinical utility as a single agent for extensive metabolizers, and possibly intermediate metabolizers, of dextromethorphan. Bupropion inhibits the metabolism of dextromethorphan, and raises the plasma concentration of dextromethorphan, and can thus improve its therapeutic efficacy. Similarly, bupropion may allow dextromethorphan to be given less often or in a lower amount, such as once a day instead of twice a day, once a day instead of three times a day, once a day instead of four times a day, twice a day instead of three times a day, or twice a day instead of four times a day, without loss of therapeutic efficacy.

The MADRS is a clinician-rated scale. The MADRS is used to assess depressive symptomatology during the previous week. Subjects are rated on 10 items to assess symptoms: 1) apparent sadness, 2) reported sadness, 3) inner tension, 4) reduced sleep, 5) reduced appetite, 6) concentration difficulties, 7) lassitude, 8) inability to feel, 9) pessimistic thoughts, 10) suicidal thoughts. Each item yields a score of 0 to 6.

The overall score ranges from 0 to 60. A score of 0 indicates the absence of symptoms, and a score of 60 indicates symptoms of maximum severity. A total score ranging from 0 to 6 indicates that the patient is in the normal range (no depression), a score ranging from 7 to 19 indicates “mild depression,” 20 to 34 indicates “moderate depression,” a score of 35 and greater indicates “severe depression.”

In some embodiments, the human being that is treated with a combination of dextromethorphan and bupropion, e.g. for a type of depression, has, or is selected for having, a MADRS score that is at least about 20, at least about 25, at least about 30, at least about 35, at least about 40, at least about 45, at least about 50, at least about 55, about 20-25, about 25-30, about 30-35, about 35-40, about 40-45, about 45-50, about 50-55, about 55-60, about 25-35, about 35-45, about 45-60, about 25-40, or about 40-60.

In some embodiments, treatment with the combination of dextromethorphan and bupropion results in the human being having a MADRS score that is reduced by at least about 10%, at least about 20%, at least about 30%, at least about 40%, at least about 50%, about 10-20%, about 20-30%, about 30-40%, about 40-50%, about 50-60%, about 60-80%, about 80-90%, or about 90-100% as compared to baseline or placebo. In some embodiments, the reduction is compared to baseline. In some embodiments, the reduction is compared to placebo. In some embodiments, the reduction is compared to the baseline before treatment.

In some embodiments, treatment with the combination of dextromethorphan and bupropion results in the human being having a MADRS score that is less than 34, about 20-34, about 7-19, about 0-6, about 30 or less, about 26 or less, about 25 or less, about 20 or less, about 19 or less, about 17 or less, about 14 or less, about 12 or less, about 10 or less, about 8 or less, about 6 or less, about 5 or less, about 4 or less, about 3 or less, about 2 or less, about 1 or less, about 0, about 7 or less, about 0-6, about 1-2, about 2-3, about 3-4, about 4-5, about 5-6, about 6-7, about 7-8, about 8-9, about 9-10, about 10-11, about 11-12, about 12-13, about 13-14, about 14-15, about 15-16, about 16-17, about 17-18, about 18-19, about 19-20, about 18-20, about 1-3, about 3-6, about 6-9, about 9-12, about 12-14, about 12-15, about 15-19, or about 15-20.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

Some patients, even after treatment with medications such as antidepressants, may have an inadequate or no response to the treatment. Treatment resistant depression (TRD), or treatment-refractory depression, is a condition generally associated with patients who have failed treatment with at least two antidepressants. Part of the diagnosis for TRD is for the patient to have had an inadequate response to treatment with the antidepressants after an adequate dose and adequate course, e.g. in the current depressive episode. TRD may be more difficult to treat due to the comorbidity of other medical or psychological illnesses, such as drug/alcohol abuse or eating disorders, or TRD being misdiagnosed. Some TRD patients have had an inadequate response to 1, 2, 3, or more adequate antidepressant treatment trials or have failed or had an inadequate response to 1, 2, 3, or more prior antidepressant treatments. In some embodiments, a patient being treated for treatment resistant depression has failed treatment with at least 1, 2, 3, 4, 5, 6, 7, 8, 9, 10, or more antidepressant therapies. In addition to other depressed patients, the combination of bupropion and dextromethorphan may be effective in treating patients suffering from treatment resistant depression with suicidal ideation.

Patients who may benefit from the treatments described herein include pediatric patients, such as patients under about 18 years of age, about 0-5 years of age, about 5-10 years of age, about 10-12 years of age, or about 12-18 years of age; adult patients, such as patients having an age of about 18-70 years, about 18-65 years, about 18-30 years, about 18-20 years, about 20-30 years, about 30-40 years, about 40-50 years, about 50-60 years, about 60-70 years, about 70-80 years, about 80-90 years, about 30-50 years, about 50-65 years; elderly patients, such as patients over 65 years of age, about 65-75 years of age, about 75-90 years of age, or over 90 years of age; and about 41 years of age or older.

In some embodiments, the human being that is treated with a combination of dextromethorphan and bupropion, e.g. for a type of depression, is, or is selected for being, of Asian descent. In some embodiments, the human being that is treated with a combination of dextromethorphan and bupropion, e.g. for a type of depression, is, or is selected for being, of Japanese descent. In some embodiments, the human being that is treated with a combination of dextromethorphan and bupropion, e.g. for a type of depression, is, or is selected for being, of Korean descent. In some embodiments, the human being that is treated with a combination of dextromethorphan and bupropion, e.g. for a type of depression, is, or is selected for being, of Chinese descent. The assignment of an individual as having Asian, Chinese, Japanese, or Korean descent may be based upon self-reporting by the individual. In these Asian individuals, the combination of dextromethorphan and bupropion may be effective for treating depression where bupropion alone is not effective for treating depression. This may be of particular importance because patients of Asian descent may suffer from more severe depression than those of other ethnic or cultural groups.

In some embodiments, the human being that is treated with a combination of dextromethorphan and bupropion, e.g. for a type of depression, is suffering from, or is selected for suffering from, a major depressive episode that has lasted between about 8 weeks and about 24 months, about 1-6 months, about 6-12 months, about 1-2 years, at least about 1 week, at least about 2 weeks, at least about 3 weeks, at least about 4 weeks, at least about 6 weeks, at least 7 weeks, at least about 2 months, at least about 3 months, at least about 4 months, at least about 5 months, at least about 6 months, at least about 9 months, at least about 1 year, at least about 18 months, at least about 2 years, about 1-12 weeks, about 3-6 months, about 6-9 months, about 9-12 months, about 12-18 months, about 18-24 months, about 2-4 years, about 4-6 years, about 6-10 years, about 10-20 years or longer.

In some embodiments, the human being that is treated with a combination of dextromethorphan and bupropion, e.g. for a type of depression, has, or is selected having, about 1-100, or more, lifetime depressive episodes, such as a major depressive episodes, including at least 1, at least 2, at least 3, at least 4, at least 5, at least 6, at least 7, at least 10, at least 15, at least 20, at least 30, at least 40, at least 50, at least 60, at least 70, at least 80, at least 90, at least 100, 1-5, 5-10, 10-20, 20-30, 30-40, 40-50, 50-60, 60-70, 70-80, 80-90, 90-100, or 4-7 lifetime depressive episodes.

In some embodiments, the human being that is treated with a combination of dextromethorphan and bupropion, e.g. for a type of depression, has, or is selected for having, an inadequate response to one or more prior antidepressant therapies, e.g. 1, 2, 3, 4, 5 or more prior antidepressant therapies, including prior antidepressant therapies in the current depressive episode (e.g. the current major depressive episode).

In some embodiments, the human being that is treated with a combination of dextromethorphan and bupropion, e.g. for a type of depression, is, or is selected for being male. In some embodiments, the human being that is treated with a combination of dextromethorphan and bupropion, e.g. for a type of depression, is, or is selected for being female.

The combination of bupropion and dextromethorphan is administered once a day or twice a day for at least one week, at least two weeks, at least three weeks, at least four weeks, at least six weeks, at least eight weeks, at least three months, at least four months, at least five months, or at least six months; and/or may be administered for up to four months, up to six months, up to one year, up to two years, or longer. In some embodiments, the combination of bupropion and dextromethorphan is administered twice a day for at least one week, at least two weeks, at least three weeks, at least four weeks, at least six weeks, at least eight weeks, at least three months, at least four months, at least five months, or at least six months; and/or may be administered for up to four months, up to six months, up to one year, up to two years, or longer. In some embodiments, the combination of bupropion and dextromethorphan is administered once a day for 1, 2, 3, 4, 5, 6, or 7 days, and then administered twice a day thereafter.

In some embodiments, at least about 50 mg, at least about 70 mg, at least about 90 mg, at least about 100 mg, at least about 110 mg, or at least about 120 mg of bupropion hydrochloride, or a molar equivalent amount of another form of bupropion (such as another salt form or the free base form) is administered once a day or twice a day. In some embodiments, the bupropion is administered once a day for 1, 2, 3, 4, 5, 6, or 7 days, and then administered twice a day thereafter.

In some embodiments, up to about 70 mg, up to about 90 mg, up to about 100 mg, up to about 110 mg, up to about 120 mg up to about 130 mg, or up to about 150 mg, of bupropion hydrochloride, or a molar equivalent amount of another form of bupropion (such as another salt form or the free base form) is administered once a day or twice a day. In some embodiments, the bupropion is administered once a day for 1, 2, 3, 4, 5, 6, or 7 days, and then administered twice a day thereafter.

In some embodiments, about 0.8× mg to about 1.2× mg of bupropion hydrochloride, or a molar equivalent amount of another form of bupropion (such as another salt form or the free base form) is administered once or twice a day, wherein x is about 50 mg (e.g. 40-60 mg), about 60 mg, about 70 mg, about 80 mg, about 90 mg, about 100 mg, about 120 mg, or about 140 mg. In some embodiments, the bupropion is administered once a day for 1, 2, 3, 4, 5, 6, or 7 days, and then administered twice a day thereafter.

In some embodiments, about 50-150 mg, about 90-120 mg, about 100-110 mg, or about 105 mg of bupropion hydrochloride, or a molar equivalent amount of another form of bupropion (such as another salt form or the free base form) is administered once a day or twice a day. In some embodiments, the bupropion is administered once a day for 1, 2, 3, 4, 5, 6, or 7 days, and then administered twice a day thereafter.

In some embodiments, at least about 30 mg, at least about 35 mg, at least about 40 mg, or about 45 mg of dextromethorphan hydrobromide, or a molar equivalent amount of another form of dextromethorphan (such as another salt form or the free base form) is administered once a day or twice a day. In some embodiments, the dextromethorphan is administered once a day for 1, 2, 3, 4, 5, 6, or 7 days, and then administered twice a day thereafter.

In some embodiments, up to about 50 mg, up to about 55 mg, up to about 60 mg, or up to about 45 mg of dextromethorphan hydrobromide, or a molar equivalent amount of another form of dextromethorphan (such as another salt form or the free base form) is administered once a day or twice a day. In some embodiments, the dextromethorphan is administered once a day for 1, 2, 3, 4, 5, 6, or 7 days, and then administered twice a day thereafter.

In some embodiments, about 0.8× mg to about 1.2× mg of dextromethorphan hydrobromide, or a molar equivalent amount of another form of dextromethorphan (such as another salt form or the free base form) is administered once a day or twice a day, wherein x is about 30 mg (e.g. 24-36 mg), about 40 mg, about 50 mg, or about 60 mg. In some embodiments, the dextromethorphan is administered once a day for 1, 2, 3, 4, 5, 6, or 7 days, and then administered twice a day thereafter.

In some embodiments, about 30-60 mg, about 40-50 mg, about 44-46 mg, or about 45 mg of dextromethorphan hydrobromide, or a molar equivalent amount of another form of dextromethorphan (such as another salt form or the free base form) is administered once a day or twice a day. In some embodiments, the dextromethorphan is administered once a day for 1, 2, 3, 4, 5, 6, or 7 days, and then administered twice a day thereafter.

Administration of the combination of bupropion and dextromethorphan may improve depression symptoms, such as the Montgomery-Åsberg Depression Rating Scale (MADRS) score. It may also reduce the Suicidality Item of the MADRS (MADRS-SI), such as by at least about 10%, at least about 20%, at least about 30%, at least about 40%, at least about 50%, at least about 60%, at least about 80%, or up to about 100%, e.g. after the combination is administered daily, such as twice a day, for one week, two weeks, four weeks, six weeks, eight weeks, or twelve weeks, etc.

In some embodiments, after eight days of receiving the combination of bupropion and dextromethorphan twice a day, the Cₘₐₓ of dextromethorphan in the human being is increased about 15- to about 25-fold as compared to administration of 60 mg of dextromethorphan hydrobromide without bupropion or as compared to administration of a single dose of the combination of bupropion and dextromethorphan.

In some embodiments, after eight days of receiving the combination of bupropion and dextromethorphan twice a day, the Cₘᵢₙ of dextromethorphan in the human being is increased about 35- to about 45-fold as compared to administration of 60 mg of dextromethorphan hydrobromide without bupropion or as compared to administration of a single dose of the combination of bupropion and dextromethorphan.

In some embodiments, after eight days of receiving the combination of bupropion and dextromethorphan twice a day, the AUC₀₋₁₂ of dextromethorphan in the human being is increased about 25- to about 45-fold as compared to administration of 60 mg of dextromethorphan hydrobromide without bupropion or as compared to administration of a single dose of the combination of bupropion and dextromethorphan.

In some embodiments, after eight days of receiving the combination of bupropion and dextromethorphan twice a day, the Cₘₐₓ of dextromethorphan in the human being is about 75-80 ng/ml.

In some embodiments, after eight days of receiving the combination of bupropion and dextromethorphan twice a day, the Cₘᵢₙ of dextromethorphan is about 42-50 ng/ml.

In some embodiments, after eight days of receiving the combination of bupropion and dextromethorphan twice a day, the AUC₀₋₁₂ of dextromethorphan in the human being is about 755 ng·hr/mL.

In some embodiments, after eight days of receiving the combination of bupropion and dextromethorphan twice a day, the Cₘₐₓ of bupropion in the human being is about 85-90 ng/ml.

In some embodiments, after eight days of receiving the combination of bupropion and dextromethorphan twice a day, the Cₘᵢₙ of bupropion is about 25-35 ng/ml.

In some embodiments, after eight days of receiving the combination of bupropion and dextromethorphan twice a day, the AUC₀₋₁₂ of bupropion in the human being is about 660-670 ng·hr/mL.

### Example 1

An open label Phase III clinical trial was conducted using a tablet containing 45 mg dextromethorphan hydrobromide and 105 mg of bupropion hydrochloride with modulated delivery. This tablet was administered twice a day to patients suffering from major depressive disorder. A total of 611 patients participated in the trial, and a total of 597 patients were treated for at least 6 months when the trial was concluded. The mean MADRS score of the total patient population was 32.7 prior to treatment. The results for the total patient population are summarized in Table 1.

TABLE 1
MADRS reduction for all patients after treatment

| Timepoint | MADRS Reduction-all patients |
|---|---|
| Baseline | 0 |
| 1 week | −10.4 |
| 2 weeks | −14.7 |
| 6 weeks | −20.6 |

A total of 37 of these patients suffered from major depressive disorder with suicidal ideation, defined as a score of ≥3 on the Suicidality Item of the Montgomery-Åsberg Depression Rating Scale (MADRS-SI). For the patients with suicidal ideation, the mean MADRS score was 36.8 and the mean MADRS-SI score was 3.4 prior to treatment. The results for these patients are summarized in Table 2.

TABLE 2
MADRS reduction for suicidal ideation patients after treatment

| Timepoint | MADRS Reduction-suicidal ideation patients |
|---|---|
| Baseline | 0 |
| 1 week | −12.9 |
| 2 weeks | −17.8 |
| 6 weeks | −22.8 |

Additionally, in the patients who suffered from suicidal ideation, the MADRS-SI score was reduced by 67.1% at the end of 1 week of treatment, 73.5% at the end of 2 weeks of treatment, and 82.4% at the end of 4 weeks of treatment as compared with baseline. This is potentially lifesaving because it shows a quick reduction in the risk of suicide.

1. A method of treating depression or reducing risk of suicide, comprising administering a combination of about 90 mg to about 120 mg of bupropion hydrochloride, or a molar equivalent amount of another form of bupropion, and about 40 mg to about 50 mg of dextromethorphan hydrobromide, or a molar equivalent amount of another form of dextromethorphan, to a human being; wherein the combination is administered twice a day to the human being suffering from major depressive disorder and having a score of 3 or greater on the Suicidality Item of the Montgomery-Åsberg Depression Rating Scale (MADRS-SI).

2. The method of claim 1, wherein the combination comprises about 105 mg of bupropion hydrochloride, or a molar equivalent amount of another form of bupropion, and about 45 mg of dextromethorphan hydrobromide, or a molar equivalent amount of another form of dextromethorphan.

3. The method of claim 1, wherein the combination is administered twice a day for at least one week.

4. The method of claim 3, wherein the combination is administered twice a day for at least two weeks.

5. The method of claim 4, wherein the combination is administered twice a day for at least six weeks.

6. The method of claim 1, wherein the MADRS-SI of the human being is reduced by at least 30% after the combination is administered to the human being twice a day for one week.

7. The method of claim 1, wherein the MADRS-SI of the human being is reduced by at least 50% after the combination is administered to the human being twice a day for two weeks.

8. The method of claim 1, wherein the MADRS-SI of the human being is reduced by at least 60% after the combination is administered to the human being twice a day for four weeks.

9. The method of claim 1, wherein after eight days of administration of the combination twice a day, the Cₘₐₓ of dextromethorphan in the human being is increased about 15- to about 25-fold as compared to administration of a single dose of the combination.

10. The method of claim 1, wherein after eight days of administering the combination twice a day, the Cₘᵢₙ of dextromethorphan in the human being is increased about 35- to about 45-fold as compared to administering a single dose of the combination.

11. The method of claim 1, wherein after eight days of administering the combination twice a day, the AUC₀₋₁₂ of dextromethorphan in the human being is increased about 25- to about 45-fold as compared to administering a single dose of the combination.

12. The method of claim 2, wherein after eight days of administering the combination twice a day, the Cₘₐₓ of dextromethorphan in the human being is about 75 ng/ml to about 80 ng/ml.

13. The method of claim 2, wherein after eight days of administering the combination twice a day, the Cₘᵢₙ of dextromethorphan is about 42 ng/ml to about 50 ng/ml.

14. The method of claim 2, wherein after eight days of administering the combination twice a day, the AUC₀₋₁₂ of dextromethorphan in the human being is about 755 ng·hr/mL.

15. The method of claim 2, wherein after eight days of administering the combination twice a day, the Cₘₐₓ of bupropion in the human being is about 85 ng/ml to about 90 ng/ml.

16. The method of claim 2, wherein after eight days of administering the combination twice a day, the Cₘᵢₙ of bupropion is about 25 ng/ml to about 35 ng/ml.

17. The method of claim 2, wherein after eight days of administering the combination twice a day, the AUC₀₋₁₂ of bupropion in the human being is about 660 ng·hr/mL to about 670 ng·hr/mL.
